(19) United States
(12) Patent Application Publication
(10) Pub. No.: US 2021/0000697 A1
(43) Pub. Date: Jan. 7, 2021

(54) METHODS FOR DENSIFICATION AND STRUCTURAL ALIGNMENT OF BIOMINERALIZED MATERIAL

(71) Applicant: NATURAL ENAMEL, LLC, Charlotte, NC (US)

(72) Inventors: Uschi M. GRAHAM, Lexington, KY (US); Stephen M. LIPKA, Lexington, KY (US)

(21) Appl. No.: 17/025,218

(22) Filed: Sep. 18, 2020

Related U.S. Application Data
(63) Continuation of application No. PCT/US19/23135, filed on Mar. 20, 2019.
(60) Provisional application No. 62/646,222, filed on Mar. 21, 2018.

(51) Int. Cl. A61K 6/30; A61L 27/12; A61L 27/36; A61L 27/38

(52) U.S. Cl. CPC A61K 6/30; A61L 27/12; A61L 27/3691; A61L 2430/12; A61L 27/3834; A61L 2400/12; A61L 27/3687

(57) ABSTRACT

A method of vacuum densification and simultaneous alignment of mineral components formed inside biomineralized organoids includes providing a pressing die system that includes a push rod arranged within a sleeve, a sample chamber, and a semi-porous support plate equipped with a vacuum pump system. A hydrated biomineralized organoid sample, including a mineral component, is inserted into the sample chamber. The biomineralized organoid sample is mechanically compressed by exerting a force via the push rod so that a solid fraction of the biomineralized organoid sample is compressed while a portion of a liquid fraction passes through the semi-porous support plate, thereby leaving the biomineralized organoid sample in a partially dehydrated state. The portion of the liquid fraction that passes through the semi-porous support plate is removed via the vacuum pump system. Mechanical compression of the solid fraction and vacuum removal of the portion of the liquid fraction facilitates an increase in density of the mineral component and an increase in alignment of particles that comprise the mineral component.

### CROSS-REFERENCE TO RELATED APPLICATIONS

For purposes of the United States, the present application is a nonprovisional patent application of, and claims priority under 35 U.S.0 § 119(e) to, U.S. Provisional Patent Application Ser. No. 62/646,222, filed Mar. 21, 2018, the entirety of which is expressly incorporated herein by reference.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE INVENTION

The present invention generally relates to biomimetic mineralization systems, and, in particular, to a biomimetic mineralization method and controlled adjustment process that leads to the alignment and simultaneous densification of biomineralized nanocrystals that are synthesized and matured in enamel organoid cultures.

Recent breakthroughs in cell biology have allowed the differentiation of various cell populations and production of cell derivatives in vitro using three-dimensional (3D) culture systems, which makes organogenesis of mineral matter enamel products possible. This process allows for the biomimetic formation of nanomineralized crystals that join together and form a hard substance consisting of greater than 90% mineral matter, with organic substances. Tooth enamel is understood to be the hardest tissue of the body, and, therefore, it is surprising that natural enamel has its origin in dental epithelial cells that have the ability to secrete hydroxyapatite-type mineral matter. FIG. 1 is a series of schematic illustrations of natural enamel with biomineralized microstructures and nanostructures. As illustrated therein, natural enamel exhibits parallel aligned mineral prisms in an organic matrix. Within each mineral prism, HA crystals are well-aligned in a generally parallel arrangement.

Forming hydroxyapatite (HA) or HA-derivatives inside a designed 3D culture system (i.e., an organoid substrate) has been of interest and has been demonstrated previously (see WO 2015/168022 A1, which involves the actual mineralization aspect of forming HA and HA-derivatives in organoids). FIG. 2A is a schematic illustration of biomimetic enamel formation using 3D cellular system organoids as a growth medium, whereby HA nanocrystallites are randomly oriented instead of aligned with one another, and FIG. 2B is a high-resolution transmission electron microscopy (HR-TEM) image showing enamel organoids with randomly-oriented HA nanocrystallites. In the mature biomineralized system illustrated in FIG. 2A, the formed HA nanocrystallites are randomly aligned with one another, which creates a material that is low in density.

Biomineralization. In the context of this invention, biomineralization involves the nucleation, precipitation and growth of HA or HA-derivatives prepared from a mineralizing solution inside a preselected organoid 3D culture material that provides structural and chemical support. Ionic solutions nucleate and form desirable mineral matter inside the biological vessel. Biomineralization entails specific as well as non-specific interactions between cell components (e.g., cell walls, proteins, enzymes, phospholipids, etc.) and inorganic components (e.g., ions, nuclei, nanoparticles etc.).

Organoids. Three-dimensional cultures grown in vitro have emerged as new self-organized tissue material frameworks (i.e., “organoids”) that can be used for controlled biomineralization efforts. These organoids, which can be propagated using in vitro experiments, have been shown to be able to acquire preferred tissue patterning and ultimately can emulate their in vivo counterparts. Recent advances in the use of organoids involve the formation of enamel-like products using the organoids as synthesis vessels loaded with calcium and phosphate ions (or any other desired nutrients to form mineral matter). Growth factors, among other additives, may be included to promote nucleation and precipitation of desired mineral matter nanoparticles inside the organoid template structure. International Application Publication No. WO 2015/168022 A1 shows that enamel organoids are spheroidal in appearance and expand by outward growth into 3D space where they meet with peripheral cells and form the extracellular matrix or growth-template for the biomineralization process. Information on growth of enamel organoids, deposition of calcium in the extracellular matrix after supply of an ion-containing nutrient solution, growth factors that can be used to accelerate mineral deposition, expression markers, and other facts related to the growth of enamel when using organoids can also be found in International Application Publication No. WO 2015/168022 A1.

In any formed (natural or synthetic) hydroxyapatite (HA) structure the hydroxyl (—OH) functional group can be substituted with fluoride, carbonate or chloride ions, which leads to apatite having marginally modified properties. All of these types of HA crystallize in the hexagonal crystal system and are incorporated into the overall enamel microstructure. The exceptional hardness and durability of enamel is the result of highly-organized and well-aligned inorganic HA crystals that, individually, have a nanorod-like shape, but are bundled together within the enamel structure such that HA crystals exist in parallel alignment with one another along the crystallographic c-axis. This nanoparticle/nanorod side-by-side arrangement results in an assembled nanocomposite prism structure that spans across micron-scale dimensions and makes up the building blocks of the enamel microstructure. High-resolution transmission electron microscopic (HR-TEM) analyses of enamel-type structure reveal that an organic matrix occurs in between the prism structures. The highly aligned biomineral nanocomposite of enamel (where the organic interstices act as shock absorbers) contributes to high crack resistance and high fatigue durability of enamel, even in an aggressive physiological environment that involves body fluids, acidity, and other challenges.

The highly aligned nature of the HA in enamel has been the focus of previous studies and has led to several biomimetic mineralization strategies. This includes cell-free studies that involve, among other approaches, hydrothermal synthesis methods, bioactive glass, surfactant-mediated growth of HA, precipitation of HA in the presence of select proteins (e.g., proline, amelogenin, gelatin, poly-dopamine, etc.), polyethylene oxide/polyacrylamide and micelle and dendrimer-based synthesis of HA, and agarose-based hydrogels. These cell-free biomimetic HA formation strategies have been demonstrated to result in enamel-like microstructures in vitro and in some in vivo applications, but they predominantly aim for potential tooth defect repair and remodeling or self-healing mechanisms. At this time, the above-noted synthesis approaches lack the application for larger tooth remodeling and replacement options. Furthermore, an important aspect of enamel-like structures involves the alignment of densely-seeded HA nanocrystals and the formation of prisms as is seen in natural enamel. In the example of an agarose hydrogel system, agarose was impregnated with both calcium and phosphate ions, and the organic substrate structure guided a precise biomimetic mineralization of elongated HA-precursor particles sandwiched within the organic matrix template. The organic template works as a space-restricting mineralization host matrix that facilitates a size-restricted and spatially-controlled formation of the desired calcium phosphate mineralization complex (i.e., HA nanoprisms), while calcified collagen fibrils of an underlying tooth helps guide the self-alignment of newly formed HA crystallites to force an ordered arrangement and strict perpendicular growth to the surface of the exposed dentin.

In the process of natural enamel formation via the organic matrix-mediated biomineralization route, the process is initiated and continues in extracellular regions where the alignment (i.e., orientation) and densification (i.e., packing) of the inorganic component crystals takes place. When the organic constituents (i.e., proteins or cell wall fragments or other organic molecules) break down or degrade, there can be a concurrent nucleation and growth of HA crystals in their place. Significant nucleation events include ion transport to the region of biomineralization and continued supply thereof, as well as nanoparticle formation and deposition (the latter of which also requires pH and ionic strength to be favorable for the precipitation of a certain phase from a solution reservoir). After enough nanoparticles are formed, continued growth is guided through surface reactions and outward radial growth. In the enamel organoid, nanoparticle formation and deposition results at first in a random orientation of the crystallites and, in some cases, a radial growth that is due to the local environmental conditions and ion diffusion parameters. As the inorganic phase (i.e., HA nanocrystals) continues to grow, the compositional ratios (organic/inorganic) lead to an overproduction of crystals (up to 99% inorganic HA crystals). Nucleation and growth of HA results first in nanorod formation, or needles, because crystal growth proceeds along the crystallographic c-axis. However, as they meet and are crowded by other nanorods, the particle growth extends in other directions (e.g., along the width of the crystallites; a-axis and b-axis).

Self-alignment of HA nanoprisms and the guided growth direction of mineralized nanocrystals is not part of the biomimetic mineralization approach that uses organoids as growth templates. However, 3D culture systems that can be used to shape enamel organoids carry the potential to generate large quantities of enamel products. The production/synthesis of large enough quantities of enamel is significant to supply biomineralized HA materials for use in connection with the manufacture of surgical dental restorations and other prostheses. The approach also can support enough material production to supply biomineralized HA for other applications, such as shark-skin type scales or large shields (for armor-type structural support) with physico-chemical properties similar to or the same as that of tooth enamel.

In nature, certain biomineralized forms of HA are extremely dense and hard, with a higher specific strength and toughness than any engineered composite materials known today. Mature natural enamel contains only a very low percentage of protein matrix (i.e., organic phase), but is hard, crack-tolerant, and abrasion-resistant. One such example involves the protective appendages of the mantis shrimp, which are made of very dense, but highly aligned, crystalline HA nanorods stacked inside microprisms. Corresponding crack propagation of the impact region is highly reduced due to the presence of thin (i.e., nanoscale) deposit layers of chitin (which is a long-chain polymer of N-acetylglucosamine, a derivative of glucose) and chitosan (which is a linear polysaccharide made of randomly distributed D-glucosamine and N-acetyl-D-glucosamine). The thin chitosan-based inter-layers aid in preventing crack propagation in case the HA mineral prisms fracture. This is the same kind of interlayer crack impedance mechanism seen in natural teeth enamel where the HA prisms of the enamel rod are surrounded/protected by thin protein-based interlayers. These organic interlayers result in an overall lower modulus or fissure growth upon impact due to a directional change in fissure propagation (similar to what occurs in different sediment layers during drilling or earthquakes), where energy is transferred or reduced differently across layers made up of materials with variances in modulus.

In view of the foregoing, a need exists for improvement in the application of 3D culture systems to provide organoids for guided biomineralization such that resultant enamel exhibits higher specific strength and toughness. This, and other needs, are addressed by one or more aspects of the present invention.

### SUMMARY OF THE INVENTION

The present invention includes many aspects and features. Moreover, while many aspects and features relate to, and are described in, the context of alignment and simultaneous densification of biomineralized nanocrystals that are synthesized and matured in enamel organoid cultures, the present invention is not limited to use only within this context and, instead, can be applied within any biomineralized system, as will become apparent from the following summaries and detailed descriptions of aspects, features, and one or more embodiments of the present invention.

Accordingly, in an aspect of the present invention, a method of vacuum densification and simultaneous alignment of mineral components formed inside biomineralized organoids comprises: providing a pressing die system that includes a push rod arranged within a sleeve, a sample chamber, and a semi-porous support plate equipped with a vacuum pump system; inserting a hydrated biomineralized organoid sample, including a mineral component, into the sample chamber; mechanically compressing the biomineralized organoid sample, by exerting a force via the push rod, so that a solid fraction of the biomineralized organoid sample is compressed while a portion of a liquid fraction passes through the semi-porous support plate, thereby leaving the biomineralized organoid sample in a partially dehydrated state; and removing the portion of the liquid fraction that passes through the semi-porous support plate via the vacuum pump system. Mechanical compression of the solid fraction and vacuum removal of the portion of the liquid fraction facilitates an increase in density of the mineral component and an increase in alignment of particles that comprise the mineral component.

In a feature of this aspect, the biomineralized organoid sample is an enamel organoid sample.

In another feature of this aspect, mechanical compression of the solid fraction and vacuum removal of the portion of the liquid fraction occurs simultaneously.

In another feature of this aspect, the pressing die system is configured so that the force generates an increasing degree of pressure upon the biomineralized organoid sample.

In another feature of this aspect, the semi-porous support plate is adapted to facilitate liquid fraction removal from the biomineralized organoid sample without reintroduction of the removed liquid fraction or any other liquid while avoiding complete dehydration.

In another feature of this aspect, removing the portion of the liquid fraction via the vacuum pump system includes vacuum removal of components added to the biomineralized organoid sample to affect at least partial dissolution of organic matrices or to affect ion exchange reactions.

In another feature of this aspect, the pressing die system further includes a pressure injection system to facilitate introduction of a liquid component comprised of one or more reagents to the partially dehydrated biomineralized organoid sample, the pressure injection system including a pressure injection valve and a fitting that connects to the sample chamber.

In another feature of this aspect, the method further comprises rehydrating the biomineralized organoid sample by introduction of the liquid component via the pressure injection system.

In another feature of this aspect, rehydrating the biomineralized organoid sample occurs simultaneously with mechanical compression of the biomineralized organoid sample.

In another feature of this aspect, the introduced liquid component includes one or more of an aqueous liquid solution, an organic liquid solution, a gel, or a deep eutectic solvent.

In another feature of this aspect, the method further comprises automatically readjusting an internal pressure of the sample chamber to accommodate for introduction of the liquid component.

In another feature of this aspect, the introduced liquid component includes a reagent solute to at least partially digest cellular membranes of the biomineralized organoid sample, thereby releasing and concentrating the mineral component from the biomineralized organoid sample for compression and alignment. In another feature of this aspect, the reagent solute includes an enzyme.

In another feature of this aspect, the method further comprises ultrasonically agitating the biomineralized organoid sample to promote fracturing cell walls of the biomineralized organoid sample so as to enhance separation of clusters of particles of the mineral component and to enhance movement of particles of the mineral component, thereby facilitating realignment of the particles in a structural arrangement.

In another feature of this aspect, the structural arrangement of the particles of the mineral component exists along an axis, whereby groups of particles are aligned in a generally parallel relationship.

In another feature of this aspect, the particles of the mineral component include hydroxyapatite nanocrystals.

In another feature of this aspect, ultrasonic agitation of the biomineralized organoid sample includes placing at least the sample chamber containing the biomineralized organoid sample in an ultrasonic bath.

In another feature of this aspect, ultrasonic agitation of the biomineralized organoid sample occurs simultaneously with a thermal treatment to increase a temperature or temperature gradient of the biomineralized organoid sample.

In another feature of this aspect, ultrasonic agitation of the biomineralized organoid sample occurs simultaneously with mechanical compression of the biomineralized organoid sample.

In another feature of this aspect, removal of the portion of the liquid fraction includes removal of a portion of an organic phase of the biomineralized organoid sample.

In another feature of this aspect, following removal of the portion of the organic phase, a remaining portion of the organic phase comprises approximately 1 wt % to approximately 5 wt % of the biomineralized organic sample.

In another feature of this aspect, the method further comprises mechanically compressing, via the force exerted by the push rod, a remaining portion of the organic phase into thin layers capable of entering into alignment with particles of the mineral component.

In another feature of this aspect, the thin layers of the organic phase are intercalated with groups of particles of the mineral component in a generally parallel relationship, thereby facilitating enhanced crack resistance of a resultant mineral-based compound.

In another feature of this aspect, the method further comprises increasing a scale of the biomineralized organoid sample in the sample chamber to support a corresponding increase in production of a resultant mineral-based compound that exhibits enhanced density and structural alignment.

In another feature of this aspect, the pressing die system utilizes a cube-shaped chamber and push-rod to facilitate formation of a mineral-based compound in the general shape of a cube.

In another feature of this aspect, the pressing die system utilizes a cylinder-shaped chamber to facilitate formation of a mineral-based compound in the general shape of a cylinder.

In another aspect of the present invention, a method of vacuum densification and simultaneous alignment of mineral components formed inside biomineralized organoids comprises: providing a pressing die system that includes a push rod arranged within a sleeve, a sample chamber, a vacuum pump system, and a pressure injection system connected to the sample chamber; inserting a hydrated biomineralized organoid sample, including a mineral component, into the sample chamber; mechanically compressing the biomineralized organoid sample, by exerting a force via the push rod, so as to partially dehydrate the biomineralized organoid sample and at least partially compact a solid fraction thereof; rehydrating the biomineralized organoid sample by introduction of a liquid component via the pressure injection system, the liquid component including a reagent solute to at least partially digest cellular membranes of the biomineralized organoid sample, thereby releasing the mineral component from the biomineralized organoid sample; ultrasonically agitating the biomineralized organoid sample to promote separation of clusters of particles of the mineral component and to enhance movement of particles of the mineral component, thereby enhancing alignment of the particles in a structural arrangement; removing at least a portion of a liquid fraction from the pressing die system, via the vacuum pump system, the liquid fraction including at least a portion of an organic phase removed from the biomineralized organoid sample and at least a portion of the liquid component introduced via the pressure injection system; heating the biomineralized organoid sample, via a controlled process using an optimized heating rate, to promote crystallization of the mineral component; and optionally repeating one or more of the mechanical compression step, the rehydration step, the ultrasonic agitation step, the liquid fraction removal step, and the controlled heating step by one or more repetitions. The mechanical compression step, the rehydration step, the ultrasonic agitation step, the liquid fraction removal step, and the controlled heating step, alone or in any combination with one another, facilitate one or more of densification of the mineral component, alignment of particles of the mineral component in a structural arrangement, enhancement of crystallization of the mineral component, and intercalation of groups of particles of the mineral component with layers of a remaining portion of the organic phase, thereby promoting formation of a densified and structurally-aligned mineral-based compound exhibiting enhanced strength and crack resistance.

In a feature of this aspect, at least two of the mechanical compression step, the rehydration step, the ultrasonic agitation step, the liquid fraction removal step, the controlled heating step, or an optional repetition of any of the foregoing steps, occur simultaneously with one another.

In another feature of this aspect, the biomineralized organoid sample is an enamel organoid sample.

In another feature of this aspect, the particles of the mineral component include hydroxyapatite nanocrystals.

In another feature of this aspect, the pressing die system is configured so that the force generates an increasing degree of pressure upon the biomineralized organoid sample.

[symbol]

In another feature of this aspect, the introduced liquid component includes one or more of an aqueous liquid solution, an organic liquid solution, a gel, or a deep eutectic solvent.

In another feature of this aspect, the method further comprises automatically readjusting an internal pressure of the sample chamber to accommodate for introduction of the liquid component.

In another feature of this aspect, the reagent solute includes an enzyme.

In another feature of this aspect, the structural arrangement of the particles of the mineral component exists along an axis.

In another feature of this aspect, the structural arrangement includes groups of particles of the mineral component arranged in a generally parallel relationship with one another.

In another feature of this aspect, mechanical compression of the biomineralized sample includes compressing the remaining portion of the organic phase into thin layers. In another feature of this aspect, one or more of the rehydration step, the ultrasonic agitation step, and the liquid fraction removal step, in combination with one another, facilitate arrangement of the thin layers into a generally parallel, intercalated relationship with the groups of particles of the mineral component.

In another feature of this aspect, the remaining portion of the organic phase comprises approximately 1 wt % to approximately 5 wt % of the biomineralized organic sample.

In another feature of this aspect, ultrasonically agitating the biomineralized organoid includes placing at least the sample chamber containing the biomineralized organoid in an ultrasonic bath.

In another feature of this aspect, ultrasonic agitation of the biomineralized organoid sample occurs simultaneously with a thermal treatment to increase a temperature or temperature gradient of the biomineralized organoid sample.

In another feature of this aspect, removal of the portion of the organic phase occurs prior to mechanical compression of the biomineralized mineral sample.

In another feature of this aspect, the method further comprises increasing a scale of the biomineralized organoid sample in the sample chamber to support a corresponding increase in production of a resultant mineral-based compound that exhibits enhanced density and structural alignment.

In another feature of this aspect, the pressing die system utilizes a cube-shaped chamber and push-rod to facilitate formation of a mineral-based compound in the general shape of a cube.

In another feature of this aspect, the pressing die system utilizes a cylinder-shaped chamber to facilitate formation of a mineral-based compound in the general shape of a cylinder.

In another feature of this aspect, the method further comprises heating the densified and structurally-aligned mineral-based compound to remove additional organic layers.

In another feature of this aspect, the method further comprises pressure injecting the densified and structurally-aligned mineral-based compound with a nutrient-rich solution, thereby filling voids left by the removed organic layers and imparting the densified and structurally-aligned mineral-based compound with an enhanced characteristic attributable to the nutrient-rich solution.

In another feature of this aspect, the densified and structurally-aligned mineral-based compound includes an organic component that comprises less than 10 wt % of the compound.

In another feature of this aspect, the densified and structurally-aligned mineral-based compound includes an organic component that comprises less than 3 wt % of the compound.

In another feature of this aspect, the densified and structurally-aligned mineral-based compound includes an organic component that comprises less than 1 wt % of the compound.

In another aspect, the present invention includes a method of vacuum densification and simultaneous alignment of mineral components formed inside biomineralized organoids, substantially as shown and described.

In another aspect, the present invention includes a mineral-based compound, formed in accordance with a method of vacuum densification and simultaneous alignment of mineral components derived from biomineralized organoids, substantially as shown and described.

In addition to the aforementioned aspects and features of the present invention, it should be noted that the present invention further encompasses the various logical combinations and subcombinations of such aspects and features. Thus, for example, claims in this or a divisional or continuing patent application or applications may be separately directed to any aspect, feature, or embodiment disclosed herein, or combination thereof, without requiring any other aspect, feature, or embodiment.

### BRIEF DESCRIPTION OF THE DRAWINGS

One or more preferred embodiments of the present invention now will be described in detail with reference to the accompanying drawings, wherein the same elements are referred to with the same reference numerals, and wherein,

FIG. 1 is a series of schematic illustrations of natural enamel with biomineralized microstructures and nanostructures;

FIG. 2A is a schematic illustration of biomimetic enamel formation using 3D cellular system organoids as a growth medium, whereby HA nanocrystallites are randomly oriented instead of aligned with one another;

FIG. 2B is a high-resolution transmission electron microscopy (HR-TEM) image showing enamel organoids with randomly-oriented HA nanocrystallites;

FIGS. 3A and 3B are each schematic illustrations of a mechanical system for facilitating densification and structural alignment of biomineralized material, in accordance with one or more aspects of the present invention;

FIG. 4 is a schematic illustration of a method of densification and structural alignment of biomineralized material in accordance with one or more aspects of the present invention

FIG. 5A is an HR-TEM image of randomly-oriented HA nanocrystallites prior to densification and structural alignment;

FIG. 5B is an HR-TEM image of partially-aligned compressed HA nanocrystallites following vacuum compression;

FIG. 5C is an HR-TEM image of structurally aligned HA nanocrystallites following vacuum compression and ultrasonic agitation;

FIG. 6 is a schematic flow chart illustrating various steps of the methods described herein;

FIG. 7A is a schematic representation illustrating aligned broken cell wall fragments that form interspaced organic divider layers between layers of crystal rods;

FIG. 7B is a schematic representation illustrating partial elimination of the organic fraction from the sample via a thermal or chemical treatment, thereby creating voids in the sample; and

FIG. 7C is a schematic representation illustrating a densified and structurally aligned sample.

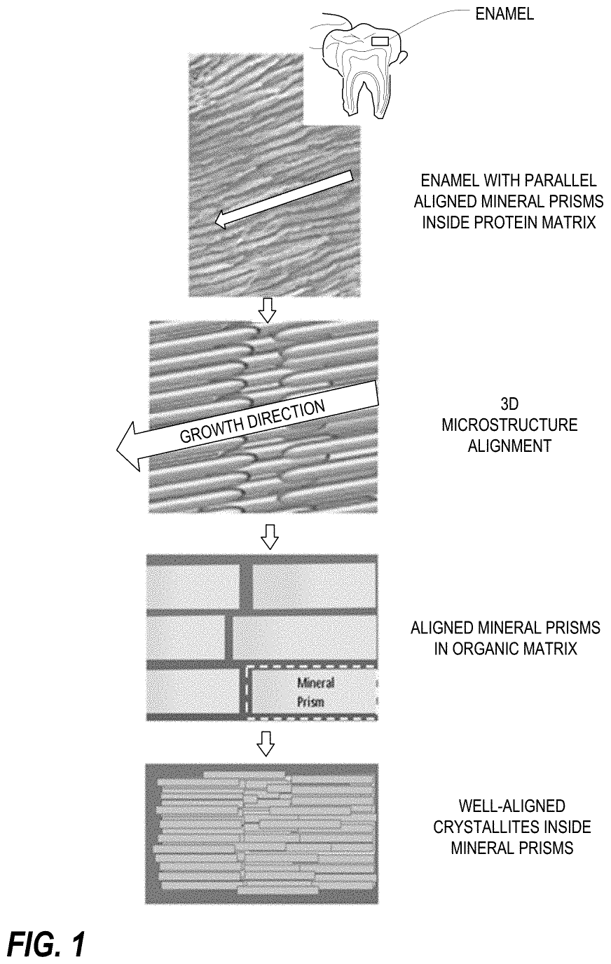

### DETAILED DESCRIPTION

As a preliminary matter, it will readily be understood by one having ordinary skill in the relevant art (“Ordinary Artisan”) that the invention has broad utility and application. Furthermore, any embodiment discussed and identified as being “preferred” is considered to be part of a best mode contemplated for carrying out the invention. Other embodiments also may be discussed for additional illustrative purposes in providing a full and enabling disclosure of the invention. Furthermore, an embodiment of the invention may incorporate only one or a plurality of the aspects of the invention disclosed herein; only one or a plurality of the features disclosed herein; or combination thereof As such, many embodiments are implicitly disclosed herein and fall within the scope of what is regarded as the invention.

Accordingly, while the invention is described herein in detail in relation to one or more embodiments, it is to be understood that this disclosure is illustrative and exemplary of the invention, and is made merely for the purposes of providing a full and enabling disclosure of the invention. The detailed disclosure herein of one or more embodiments is not intended, nor is to be construed, to limit the scope of patent protection afforded the invention in any claim of a patent issuing here from, which scope is to be defined by the claims and the equivalents thereof It is not intended that the scope of patent protection afforded the invention be defined by reading into any claim a limitation found herein that does not explicitly appear in the claim itself

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the invention. Accordingly, it is intended that the scope of patent protection afforded the invention be defined by the issued claim(s) rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

With regard solely to construction of any claim with respect to the United States, no claim element is to be interpreted under 35 U.S.C. 112(f) unless the explicit phrase “means for” or “step for” is actually used in such claim element, whereupon this statutory provision is intended to and should apply in the interpretation of such claim element. With regard to any method claim including a condition precedent step, such method requires the condition precedent to be met and the step to be performed at least once during performance of the claimed method.

Furthermore, it is important to note that, as used herein, “a” and “an” each generally denotes “at least one”, but does not exclude a plurality unless the contextual use dictates otherwise. Thus, reference to “a picnic basket having an apple” describes “a picnic basket having at least one apple” as well as “a picnic basket having apples”. In contrast, reference to “a picnic basket having a single apple” describes “a picnic basket having only one apple”.

When used herein to join a list of items, “or” denotes “at least one of the items”, but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers”. When used herein to join a list of items, “and” denotes “all of the items of the list”. Thus, reference to “a picnic basket having cheese and crackers” describes “a picnic basket having cheese, wherein the picnic basket further has crackers”, as well as describes “a picnic basket having crackers, wherein the picnic basket further has cheese”.

Referring now to the drawings, one or more preferred embodiments of the invention are next described. The following description of one or more preferred embodiments is merely exemplary in nature and is in no way intended to limit the invention, its implementations, or uses.

Described herein are methods of densifying and aligning biomineralized nanocrystallites of enamel products that are generated using 3D cell culture systems. The use of 3D culture systems to generate enamel products via enamel organoids is described in International Application Publication No. WO 2015/168022 A1 and U.S. Application Publication No. US 2017/0035661 A1, each of which is incorporated herein by reference. The biomimetic mineralization system involves use of organoids and mineralizing solutions, which include at least calcium and phosphate-based ionic components, to form nanograins and nanocrystallites. In at least some contemplated embodiments, the formed nanograins and nanocrystallites include hydroxyapatite (HA) or equivalent mineral substitutes.

The methods described herein involve the alignment and simultaneous densification of biomineralized nanocrystallites that are synthesized and matured in enamel organoid cultures. The methods yield a structurally-oriented, densely packed and parallel-aligned and stacked nanocomposite, where nanocrystals become aligned and include a fraction of organic material (e.g., cell wall fragments). In the case of HA, alignment of HA crystals is a factor in the development of enamel properties in the nanocomposite. The organic cell fragments are arranged in a generally parallel relationship with the HA, but at lower concentrations. The resultant nanocomposite exhibits properties that compare favorably to hard enamel and that can be formed/shaped for use across a range of end-use applications. In this regard, it is contemplated that nanocomposites produced in accordance with one or more of the methods described herein are capable of use as a restorative dental product, a bone scaffold, or a skeletal prosthesis (for replacing portions of other bones that have been impaired by disease and/or trauma). Other end-use applications of the organoid-derived enamel products include, but are not limited to, protective coatings, coverings, scales, protective shields, and/or dermal denticles (i.e., shark skin types).

The alignment and intercalation processes described herein include both an inorganic phase and at least a portion of an organic phase. Generally, a mechanical process is used to facilitate vacuum densification and simultaneous alignment of biomineralized nanocrystallites. Vacuum densification is combined with a pressure injection step to reintroduce a liquid phase that allows reagents to interact with the partially-densified phase or allow slurry formation inside a compression die apparatus to force a greater alignment of nanocrystallites. These steps can be further enhanced by ultrasonic treatment prior to or during continued densification steps and thermal treatment either during or after densification. The resulting micro- and nanostructural composites mimic the aligned periodic structures of natural biomineralized materials without guided growth mechanisms. The guided growth mechanisms described herein (e.g., alignment and intercalation of organic layers with inorganic nanocrystallites) can be achieved by the combination of mechanical compression, vacuum densification, pressure injection and slurry phase alignment/compression of nanocrystallites, and a residual organic phase that is arranged relative to the inorganic phase as generally parallel interlayers.

Natural enamel is very hard yet has excellent resistance to fracture (i.e., high flexibility and hardness). This can be attributed to the structural architectures of the inorganic and organic components that are biologically guided. The methods described herein involve the use of a biomimetic growth medium to grow enamel organoids having a composition of greater than 90% mineral matter (e.g., HA nanocrystallites) with organic substances occupying the remainder. The structural micro- and nano-architecture (i.e., the alignment of nanocrystallites and interspaced organic layers) of the resultant enamel is a result of one or more of the engineering methods described herein. The methods described herein enable the mechanical alignment and densification of the inorganic phase and, at least to some degree, also that of the organic phase, in order to superimpose the properties of high flexibility and hardness that are inherent to natural enamel upon the nanocomposite. Furthermore, because a greater degree of crystal alignment can result in reduced light scattering, the methods described herein can facilitate formation of nanocomposites exhibiting greater translucency.

Steps of the methods described herein are implemented to obtain well-aligned and densified mineral particles (e.g., HA nanorods) with organic matrix layers intercalated in the same or similar stacking direction as the mineral particles. The degree of densification, alignment and amount of intercalated organic matrix components can each be modified, as might be desired, in order to support formation of nanocomposites exhibiting preferred physiochemical properties of the resultant products. In a preferred embodiment, the inorganic material includes HA nanorods, which, when aligned and coordinated with an intercalated organic matrix, can be used to generate a biomimetic enamel product.

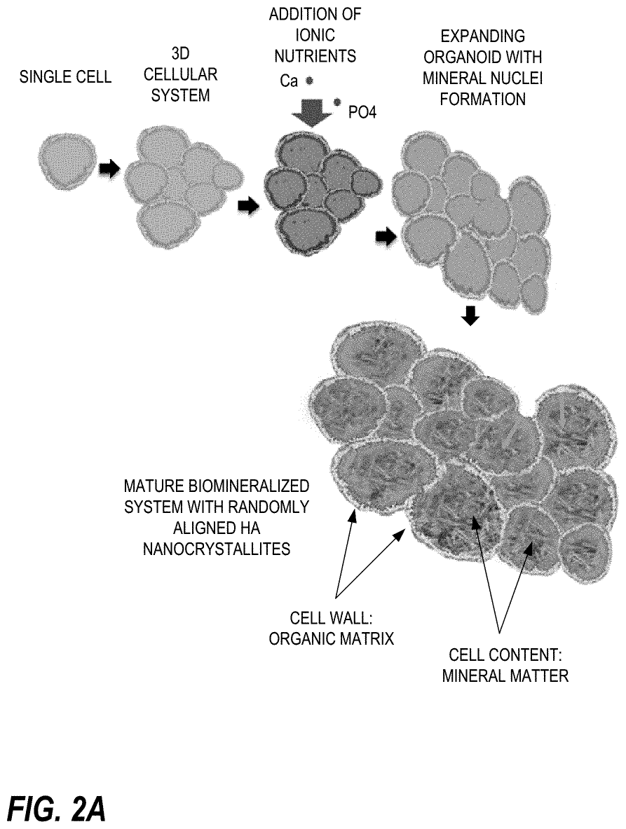

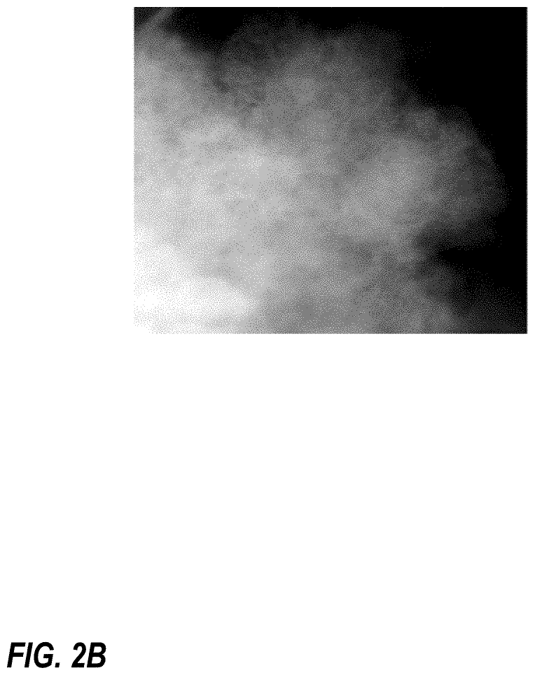

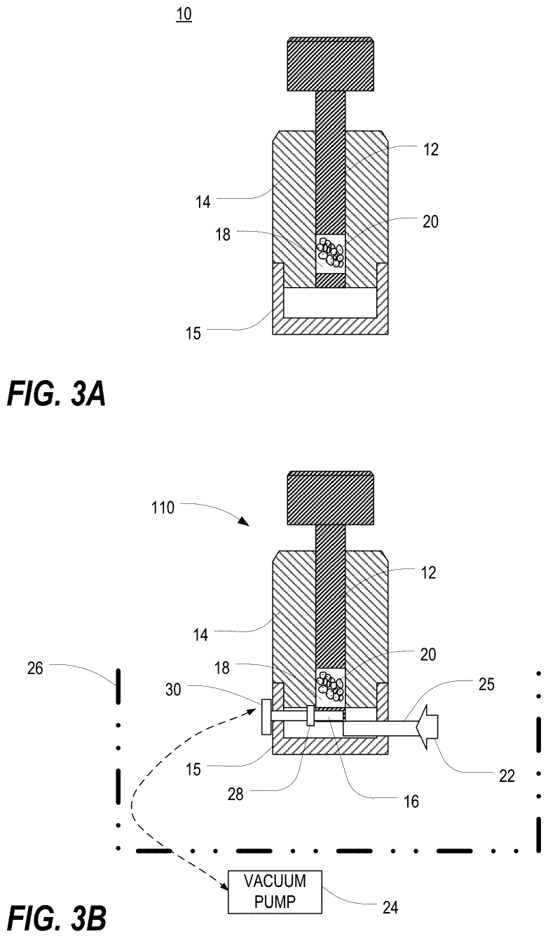

Densification and Structural Alignment. After the growth phase in the 3D cellular system, biomineralization in the organoids is complete. As shown in FIG. 2A, mature organoids have a round, oval or semi-spherical shape. However, the organoids are also highly flexible in shape and volume due to the nature of the organic cell membrane and the high liquid content inside the spheroids. As a result, mature organoids can be fitted into dies and pressed into desirable shapes, such as cylinders, cubes, or other shapes. In this regard, FIGS. 3A and 3B are each schematic illustrations of a mechanical system for facilitating densification and structural alignment of biomineralized material, in accordance with one or more aspects of the present invention. With reference to FIG. 3A, fully-hydrated organoids 20 are placed inside a sample chamber 18 of a pressing die system 10 that is outfitted with a pressure-inducing push rod 12 configured to be maneuverable within a metal sleeve 14. The pressure-inducing push rod 12 is capable of exerting a desired external force upon the organoid samples 20 arranged in the sample chamber 18. The base 15 of the pressing die system 10 can be used to support/lift the sample and can ultimately facilitate ejection thereof. Pressing the mature organoids in this manner partially or fully compresses the organoids 20 and helps to densify the biomineralized enamel materials. In at least some embodiments, the pressing die system 10 facilitates thermal treatment of the biomineralized organoid sample.

With reference to FIG. 3B, another pressing die system 110, similar to that of FIG. 3A, includes a support plate 16 arranged adjacent to the sample chamber 18. In a contemplated embodiment, the support plate 16 is semi-porous in order to facilitate removal of moisture from the organoid samples 20 while avoiding complete dehydration. In this regard, the pressing die system 110 can be used to mechanically compress the solid faction of the organoid samples 20 while a portion of the liquid phase is permitted to exit through the semi-porous plate 16, thereby partially dehydrating the samples. It is contemplated that the pressure-inducing push rod 12 can be used to exert an increasing degree of pressure (i.e., load) upon the hydrated organoid samples 20. In other contemplated embodiments, the support plate is made of a non-porous material.

In addition to the foregoing, it is contemplated that the hydrated organoids 20 in the pressure die sample chamber 18 can be subjected to a partial vacuum extraction of the liquid phase via a vacuum system. With further reference to FIG. 3B, the vacuum system is equipped with a vacuum-fitting nozzle 30, coupled with the support plate 16, and a vacuum pump 24 connected to the nozzle 30. The vacuum system can be further equipped with a semi-permeable filter 28 to facilitate moisture removal. Generally, vacuum removal of the liquid phase allows moisture from the organoid samples 20 to be removed while also compressing the sample, thereby increasing mineral density and helping to partially purify the sample. In this respect, vacuum extraction includes partial removal of moisture, liquids or gels, or that of specially-added solutions, including enzymes, that may have been added to the organoid samples. Specially-added solutions might include solutions added via a pressure injection valve to affect the digestion or at least partial dissolution of organic matrices, to break down the spheroid structure of the organoids and release the mineral content, or to affect ion exchange reactions (for fluorine treatments or other purposes). During controlled vacuum extraction of such solutes, crystallization and self-alignment of mineral particles (e.g., HA nanorods) can be guided. Though sometimes discussed herein within the context of a biomineralized system for enamel, it is contemplated that vacuum densification and simultaneous alignment of mineral components can involve any kind of biomineralized system.

Rehydration. It is contemplated that, in order to avoid complete dehydration of the organoids, not all liquid materials need be extracted early in the process. This can help to avoid breakage of brittle mineral matter that is not yet aligned or aid in the realignment by providing mobility. It is further contemplated that the organoids may be rehydrated at any time, such as by pressure injection of a liquid, in order to help promote further realignment of nanocrystals. Rehydration might also include introduction of enzymes or reagents to help partially digest the cellular membranes.

In one contemplated embodiment, a rehydration step involves pressure injection of a liquid phase to the biomineralized sample via a pressure injection system. With reference to FIG. 3B, a pressure injection system includes a pressure injection valve 22 and a fitting 25 that connects to the sample chamber 18. The injected liquid phase helps to manipulate the partially compressed and dewatered enamel sample to rehydrate/expand the sample. It is contemplated that the internal pressure of the sample chamber 18 can be automatically re-adjusted to make space for extra liquid volume entering the compression column and mixing/homogenizing with the sample. In various embodiments, the pressure-injected medium includes one or more of a liquid, a gel, or a deep eutectic solvent. It is further contemplated that the pressure-injected medium can be organic in nature and/or can exist in an aqueous form and/or includes one or more reagents. In at least some embodiments, the introduced liquid component includes a reagent solute to at least partially digest cellular membranes of the biomineralized organoid sample, thereby releasing and concentrating the mineral component from the biomineralized organoid sample for compression and alignment.

Ultrasonic Treatment. With further reference to FIG. 3B, ultrasonic agitation can be used to promote arrangement of mineral particles (e.g., HA nanorods), which are still dispersed inside a hydrated medium. Ultrasonic treatment helps to separate individual nanorods that may be stuck to one another and provides effective particle movement in the slurry stage. As shown in FIG. 3B, it is contemplated that ultrasonic agitation of the pressing die system 110 encompasses arrangement of the sample chamber 18, including the organoid samples, enamel phases, reagents, and other contents present therein, in an ultrasonic bath 26.

Ultrasonic agitation promotes the mineral particles (e.g., HA nanorods) that are formed in agglomerates, clusters, and networks, to separate from one another. Ultrasonic agitation also promotes fracturing of organoid cell walls to enhance the separation of clusters of particles of the mineral component and to enhance movement of particles of the mineral component, thereby facilitating realignment of the particles in a structural arrangement. Furthermore, ultrasonic agitation also facilitates enhanced kinetic energy in the hydrated slurry stage, via particle movement, which also helps to allow particles (e.g., HA nanorods inside nodules) to separate and realign. In various embodiments, it is contemplated that ultrasonic treatment can be performed with or without heat treatment.

In consolidation/densification experiments, an increase in particle orientation can be shown with increasing load. However, most orientation of nanocrystallites into a generally parallel alignment occurs during the very early stages of loading, while the nanocrystallites are still dispersed in a slurry-type fashion. It is contemplated that mechanical orientation and densification of the nanocrystallites can be conducted simultaneously or alternately with vacuum extraction procedures. It is further contemplated that consolidation, vacuum extraction, rehydration (i.e., re-introduction of a liquid phase), and ultrasonic treatment can be accomplished in any sequence and with any quantity of repetition until a preferred alignment of mineral particles (e.g., HA nanorods) is achieved. In this manner, densification and ultimate alignment of particles can be optimized to support a specific objective.

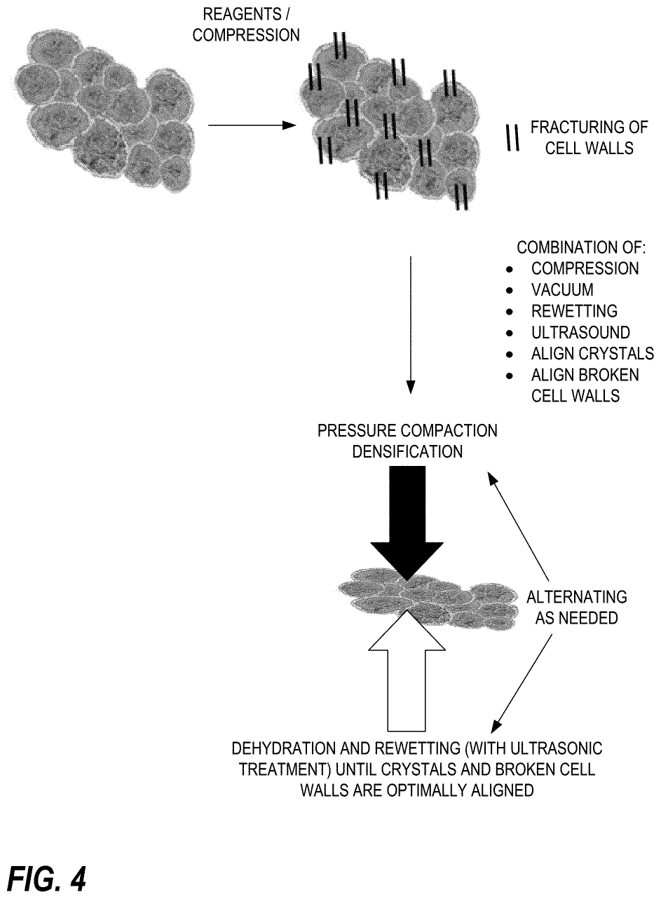

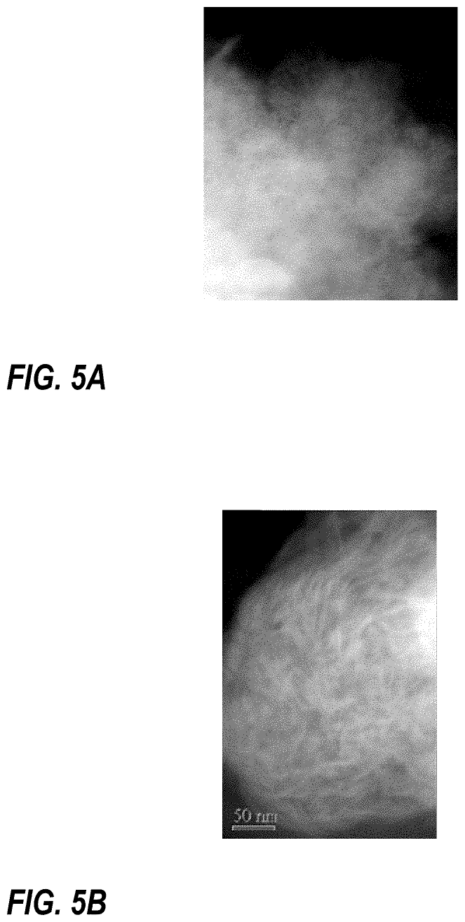

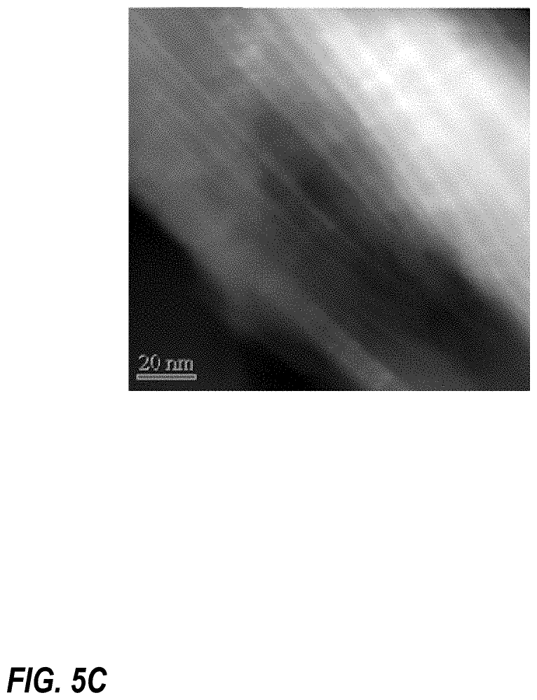

Methods as described herein utilize several runs of vacuum dewatering/dehydration, followed by cycles of rewetting that is performed in parallel with dynamic compaction. This sequence helps to optimize particle alignments as parallel bundles or inorganic material are formed. In combination, these steps help to generate negative force (e.g., vacuum extraction) and positive force (e.g., rewetting using some optimum injection forces/fluid pressure) to facilitate: (i) improved particle alignment; (ii) pore pressure dissipation (whereby some pores are difficult to collapse without pore pressure dissipation because of rigid crystallites that can poorly align and form cavities); and (iii) optimum consolidation and densification effects (whereby the better the crystallites are aligned in parallel, the greater the consolidation and densification can be in the resultant biomimetic product). At a high level, various steps of the processes described herein are detailed in FIG. 4, which is a schematic illustration of a method of densification and structural alignment of biomineralized material in accordance with one or more aspects of the present invention. Reference is also made to FIGS. 5A-5C to illustrate the effects of the densification and alignment procedures described herein. FIG. 5A is an HR-TEM image of randomly-oriented HA nanocrystallites prior to densification and structural alignment. As shown therein, nanocrystallites are unsystematic and disorderly and in disarray relative to one another. FIG. 5B is an HR-TEM image of partially-aligned compressed HA nanocrystallites following vacuum compression. Here, nanocrystallites have become more dense and begin to show signs of parallel alignment. FIG. 5C is an HR-TEM image of structurally aligned HA nanocrystallites following vacuum compression and ultrasonic agitation. Here, nanocrystallites are well-aligned and are oriented in a generally parallel relationship with one another. With further reference to FIG. 4, a combination of one or more of compression, vacuum densification, re-wetting, ultrasonic treatment, and reagent treatment to digest cellular walls can be implemented to achieve a desired densification of alignment of a biomineralized structure, such as in FIG. 5C. Furthermore, it is contemplated that compression cycles upon the sample can be alternated with cycles of dehydration and rehydration in concert with ultrasonic treatment (as depicted in FIG. 4).

In view of the foregoing, a method of particle alignment in accordance with the present invention can be based on a combination of: (i) controlled vacuum extraction of solutes; (ii) reintroduction of a liquid phase using the pressure injection to disperse particles, followed again by vacuum extraction, and (iii) ultrasonic agitation to further enhance particle dispersion and realignment. When implemented in combination with one another, these steps promote realignment of mineral nanocrystals along a preferred axis (such as the c-axis in the case of HA), parallel alignment of groups of nanocrystals, and alignment of organic matrix fragments or protein structures with the nanocrystals.

Dimensions and scale of the enamel products produced in accordance with the methods described herein depend, at least in part, on the method and tool design. However, it is contemplated that any of the methods and sequences of steps described herein can be scaled to allow for larger volume production. In this regard, it is further contemplated that the enamel organoids can themselves be scaled to accommodate larger volume production of densified and aligned material.

Intercalation of Organic Layers. Along with the parallel alignment of mineral nanorods/nanocrystals, equally important is the intercalation and insertion or layering of organic medium in some structured fashion to separate bundles of parallel-oriented crystallites. It is contemplated that the organic medium may include cell wall fragments, proteins, or other organic materials. With respect to the structuring of layers, some periodicity may be desired.

Intercalation of organic layers involves removing a desired fraction of the organic matrix derived from the cellular material (e.g., cell walls of the organoids) as well as any other organic substance that may have been used to promote growth of the organoids (e.g., enamel-forming proteins and nucleic acids, enzymes, growth factors, etc.). Following removal, the remaining organic phase occupies a low percentage (usually between approximately 1-5 wt % of the sample). This remaining organic phase can be compressed and elongated into thin layers that are intercalated with the inorganic phase. As formed, the organic thin layers are formed in a generally parallel relationship with inorganic materials (e.g., platelets, prisms, or nanorods).

Partial removal of organic material may be accomplished prior to the compaction and spatial alignment procedures and may include select chemical methods to partially extract, dissolve, or digest some of the organic fractions. In at least some embodiments, it is contemplated that the removal of organic material at this stage does not include thermal treatment (where thermal treatment might include heating to temperatures in excess of 95° C.). At this early stage, such thermal treatment can cause accelerated dehydration of the organoid, followed by reduction in malleability of the organoid product.

Once the nanocrystals inside the organoids are voided of excess surrounding moisture, the nanocrystals generally lose their ability to self-align and rearrange in space as a function of external pressure. Self-alignment of nanocrystals into elongated ribbons or rods, and particularly the parallel stacking of such ribbons or rods, is one of the hallmarks of enamel materials, and rapid dehydration tends to preserve a random orientation of the nanocrystals and prevent any kind of parallel alignment. Randomly-oriented nanocrystals in dehydrated cellular vesicles freeze into rigid networks with significant void space between intersecting crystals, and the voids can remain even after repeated densification attempts using applied external pressure. Though this kind of void space can be minimized when the rigid nanocrystal domains crumble under the applied pressure and form ultrafine fragments that fill the void spaces, such activity leads to an enamel product with very low overall hardness and strength.

Organic interlaying between nanocrystal bundles provides a different modulus and positively affects stress or crack propagation in enamel and other mineral structures by functioning as shock absorbers. The parallel insertion of organic layers in between inorganic nanocrystallite bundles is achieved by integrating and combining the use of vacuum dewatering with a controlled reintroduction of any cell membrane disrupting agents or protein dissolving agents to promote partial dissolution of the cell wall and/or fracturing of the cell walls. With reference to FIG. 3B, it is contemplated that any such agents can be reintroduced via the pressing injection valve 22 of the die pressing system 110.

This can be followed by further compaction and dewatering, steps which may need to be repeated several times. This dynamic compaction technique can make only certain parts of the spheroid-shaped cellular walls available so that the organic medium can be stacked into the spatially-arranged mineral bundles. Only a small fraction of the original organic components are needed to make thin films, and a controlled partial removal of the digested organic matrix can be extracted using a vacuum extraction procedure, as described earlier, while the material is further compacted. Compaction flattens the residual organic medium into sheets or layers. To approach a more homogeneous dispersion of the organic layers and introduce some periodicity (such as what is seen in natural tooth enamel), the above-described steps can be combined together. Some steps may need to be repeated more frequently and the order of the steps may need to be adjusted, depending on the physicochemical properties of the organic cellular materials and any organic additives (e.g., proteins, fats, nucleic acids, enzymes, growth factors, etc.). This makes the methods described herein a dynamic approach that provide the ability to disperse, align, dissolve, realign, densify, and shape the mineral materials into a biomimetic structure.

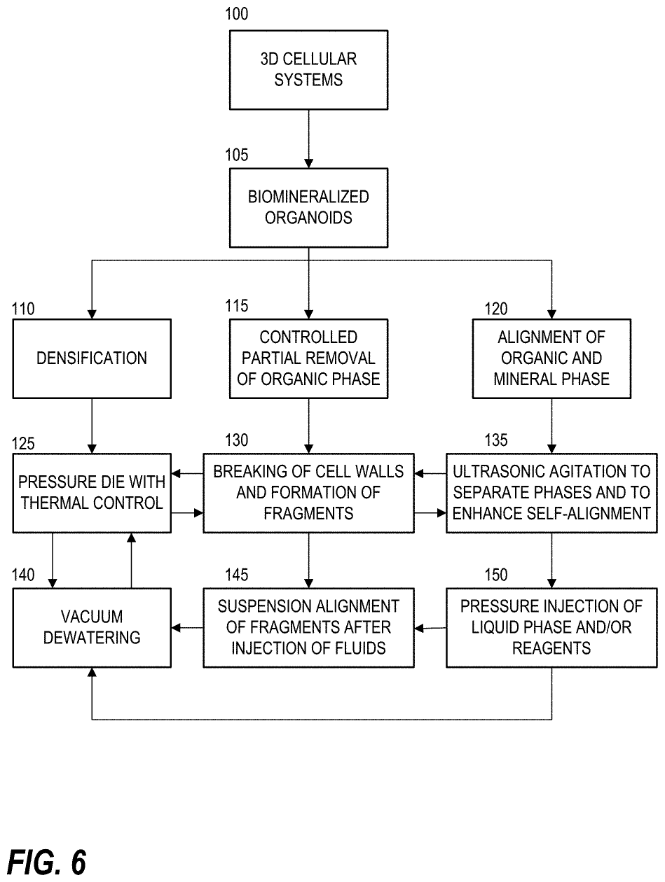

In this regard, FIG. 6 is a schematic flow chart illustrating various steps of the methods described herein. As depicted in FIG. 6, methods for densification and structural alignment of biomineralized material involve a dynamic approach, where steps are repeated and/or performed in combination with other steps to achieve a desired result. Biomineralized organoids 105 developed using a 3D cellular system 100 can be densified 110, via a pressure die with a thermal control feature 125, and aligned 115 so that the organic and mineral phases are compact and in generally parallel alignment. In some embodiments, a portion of the organic phase can be removed 120 to complement the effects of densification and structural alignment. Removal of a portion of the organic phase 120 can be accomplished by using reagents to digest or break cellular walls 130. The sample can be ultrasonically agitated 135 in furtherance of the effort to separate and align the organic and mineral phase 120. Additionally, a liquid phase can be reintroduced to the sample via pressure injection 150. Here, the sample can be rehydrated to promote further alignment of the mineral phase 145, and additional reagents can be used to further break down the organic phase. Vacuum dewatering 140 can be used to remove the reagents as well as a portion of the organic phase of the sample. It is contemplated that the steps of mechanical compression 125, ultrasonic agitation 135, rehydration 130,150, and vacuum dewatering 140 can be used in concert with another, with each step repeated as many times as might be necessary, in order to arrive at a densified and structurally aligned biomineralized material (e.g., a biomimetic enamel structure).

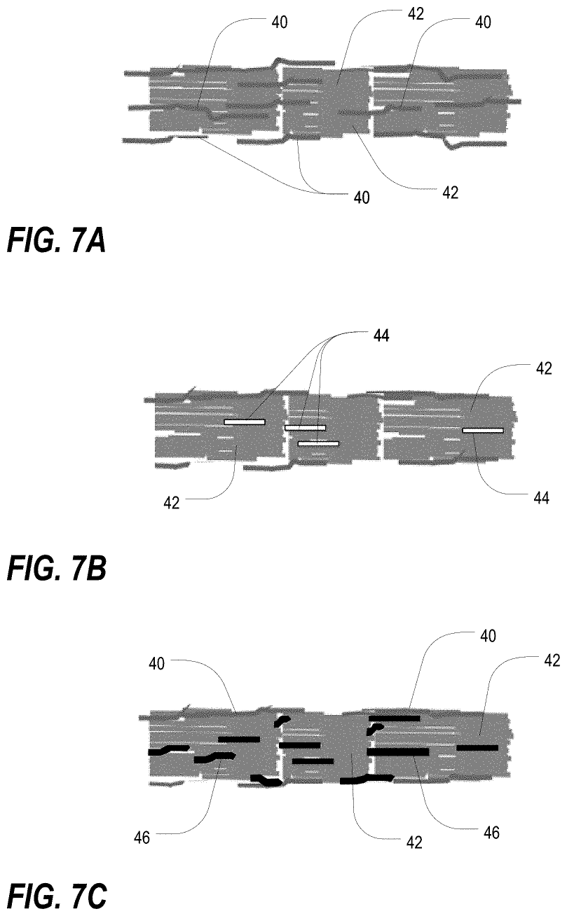

The resultant products include a residual organic matrix from the cellular membrane of the organoids that is intercalated with the inorganic nanorods and prisms. It is contemplated that the residual organic matrix follows the same or similar directional alignment as the inorganic crystallites (e.g., HA nanorods) after being fractured, partially dissolved, compressed, and realigned. In its remodeled form, the organic fraction functions as a thin, compressed interlayer medium that separate bundles of aligned nanocrystals so that the end-result biomimetic products are made of densely packed, but spatially/structurally aligned, crystalline nanorods and prisms. A schematic representation of this effect is provided in FIG. 7A, which illustrates aligned broken cell wall fragments 40 that form interspaced organic divider layers between layers of crystal rods 42. Some of the fragments rise to the surface of the sample and can be removed, while the remaining fragments form thin organic layers following compression and alignment. The intercalated organic matrix interlayer helps to establish an overall lower modulus or crack growth upon external impact force. The residual organic layers transfer stress differently compared to the brittle crystalline regions that generally impose an interlayer crack impedance mechanism.

After consolidation, whereby the nanocrystals are well-aligned and a portion of organic matrix interlayers are stacked between bundles of aligned nanocrystals, it is contemplated that the sample can optionally be heated to further remove organic fractions. The heating process can be conducted separately in an oven, which usually results in a void space being formed in the biomimetic product as organic material is removed. Or, in another contemplated embodiment, the heating process can be conducted in a die outfitted with appropriate heating coils to provide the option of simultaneous removal of organics and further compaction. A schematic representation of this effect is provided in FIG. 7B, which illustrates partial elimination of the organic fraction from the sample via a thermal or chemical treatment, thereby creating voids 44 in the sample. It is contemplated that heating the sample can be accomplished via a controlled process using an optimized heating rate.

Following the removal of additional organic layers, it is further contemplated that, in place of compaction, the material can be pressure-injected with a nutrient solution that fills the newly created void space and nucleates and grows additional mineral nanoparticles that can further improve the properties of the biomimetic product. A schematic representation of this effect is provided in FIG. 7C, which illustrates a densified and structurally-aligned sample. Here, a mineral solution has been pressure-injected to the sample to increase mineral crystallization 46 in void spaces left by dissolved organic components, thereby increasing the final density of the sample.

The biomimetic mineral products as described herein can, therefore, have a highly variable, but at the same time highly controllable organic content. In various contemplated embodiments of the designed product, the organic phase may comprise more than 10 wt %, less than 10 wt %, less than 3 wt %, or substantially no organic matter (i.e., less than 1 wt %). The layering process after consolidation (including the organic content and the density of the structure) is significant to achieving various desired physicochemical properties (e.g., hardness, modulus, etc.). Such properties are also, in turn, dependent upon the symmetry of the organized nanocrystals and organic layers. In natural enamel, the precisely organized architecture of the enamel is thought to be based on the cellular movements and their interactions with proteins, enzymes and other molecular components and mineralizing substrates, which are dependent on a complex set of gene expressions to trigger responses. In the present invention, methods as described herein involve a dynamic process with an orchestrated interplay of cellular material, mineralized nanocrystals, additions and/or removals of select components (organic and/or inorganic) as well as the reintroduction of either the same components or modified components, or the infusion of new components, into the starting system, all of which can be implemented to form an effective and comparable biomimetic enamel structure.

In at least some embodiments, it is contemplated that the aligned/densified biomineralized nanocomposite has an internal alignment structure that can span the length of the produced structure, and that the composite can be shaped with the assistance of CAD/CAM technology. In at least some other embodiments, it is contemplated that the aligned/densified biomineralized nanocomposite can exist as granular powder made up of individual grains (e.g., enamel grains), where each powder grain has its own aligned and dense nanostructure. Powder-forming applications, including methods by which the biomimetic material is formed as free-flowing grains, can be used with appropriate binders or with 3D printing applications, and such aspects are also within the scope of the present invention.

The next level of order in the biomimetic products involves the interwoven arrangement of the prisms. Packing of the prisms is probably the result of an orchestrated receding of the ameloblast cell layer. The final product is an approximately 2.5 mm thick mineralized tissue that is translucent and varies in color from yellowish-white to grayish-white. The achievement of such precisely organized architecture lies not only in the cell movement, but also in the highly controlled expression of proteins and enzymes, and the manner in which these organic molecular components interact with each other, with cell surfaces, and with the forming mineral.

Structure Shaping. Flexible manufacturing using the principles explained above with the option to integrate computer control (to determine appropriate sequences of steps, repetitions, exclusions of steps, etc.) can help to optimize the internal structure of the biomimetic material. The outer shape of the material, such as, for example, a cube (e.g., approximately 1 cubic centimeter) or a cylinder (e.g., approximately 1 cubic centimeter x cm length of the cylinder), can be obtained by predetermining the design, shape, dimensions, and volume of the die in the above-described processes. In this manner, shaped products (e.g., cubes, cylinders, etc.) can be produced that are intended for CAD/CAM milling methodology, as is done for conventional dental ceramic blocs.

It is further contemplated that dies may be 3D-printed in the shape of a desired object. Contemplated object shapes include, but are not limited to, teeth or prostheses shields (e.g., armor plates). Within the context of biomimetic enamel, it is contemplated that a still-pliable organoid enamel with highly aligned HA nanodomains and organic interlayers may be pressure-injected into such a 3D-printed die. Such 3D-printed shaped dies still should provide the option of vacuum dewatering and densification as outlined above after the pressure injection of the enamel material. This can help avoid the need for using interlocking pieces when developing an enamel product designed for the use of larger surgical bone replacements or even larger objects like protective body armor. A die injection apparatus as described herein (shaped using 3D-printing technology or unshaped using typical die shapes like cubes and cylinders) as well as the processes involved in designing such injection dies that can be used in tandem with vacuum dehydration and compaction of the injected materials (including the methods of making them, such as manufacturing 3D-printed dies that can be substituted for cubes and cylinders in the vacuum extraction and compactions system outlined above) are within the scope of the present invention. In this regard, it is contemplated that the sample chamber and/or the push-rod can be shaped (e.g., cube-shaped or cylinder-shaped) to facilitate formation of a mineral-based compound having a preferred shape.

If 3D-printed injection dies cannot be prepared for certain shapes and sizes that allow organoids to be pressure injected into the dies, it is contemplated that finished biomimetic products may be granulated/powderized to prepare powders that can be submitted to pressure injection molding using appropriate liquid media or binders that can be removed later. This application is also applicable to tissue engineering that uses scaffolds with abundant pores for pressure-injecting powder slurries.

It is contemplated that the mechanical properties of formed biomimetic enamel products can be evaluated with a nano-indentation technique. One such technique utilizes the Nano Indenter G200, which is manufactured by Agilent Technologies, headquartered in Santa Clara, Calif., USA. In one contemplated technique, at least ten points should be analyzed on selected specimens to obtain surface indentations that can be analyzed and evaluated in comparison with the nano-hardness of standard enamel products. The elastic modulus (which is a function of the location/placement and concentration of the organic phase in the enamel) should be recorded and compared to standards. The nano-indentation (loading, peak load holding, and unloading) should be conducted with industry standard time periods, which typically involve approximately 20 seconds at loading, approximately 15 to 25 seconds at peak load holding, and another approximately 20 seconds at unloading. It is contemplated that a maximum applied force (applied during loading and unloading) should be approximately 0.10 N.

Based on the foregoing description, it will be readily understood by those persons skilled in the art that the present invention has broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention. Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

What is claimed is:

1. A method of vacuum densification and simultaneous alignment of mineral components formed inside biomineralized organoids, the method comprising:
providing a pressing die system that includes a push rod arranged within a sleeve, a sample chamber, and a semi-porous support plate equipped with a vacuum pump system;
inserting a hydrated biomineralized organoid sample, including a mineral component, into the sample chamber;
mechanically compressing the biomineralized organoid sample, by exerting a force via the push rod, so that a solid fraction of the biomineralized organoid sample is compressed while a portion of a liquid fraction passes through the semi-porous support plate, thereby leaving the biomineralized organoid sample in a partially dehydrated state; and
removing the portion of the liquid fraction that passes through the semi-porous support plate via the vacuum pump system;
wherein mechanical compression of the solid fraction and vacuum removal of the portion of the liquid fraction facilitates an increase in density of the mineral component and an increase in alignment of particles that comprise the mineral component.

2. The method of claim 1, wherein the biomineralized organoid sample is an enamel organoid sample.

3. The method of claim 1, wherein mechanical compression of the solid fraction and vacuum removal of the portion of the liquid fraction occurs simultaneously.

4. The method of claim 1, wherein the pressing die system is configured so that the force generates an increasing degree of pressure upon the biomineralized organoid sample.

5. The method of claim 1, wherein the semi-porous support plate is adapted to facilitate liquid fraction removal from the biomineralized organoid sample without reintroduction of the removed liquid fraction or any other liquid while avoiding complete dehydration.

6. The method of claim 1, wherein removing the portion of the liquid fraction via the vacuum pump system includes vacuum removal of components added to the biomineralized organoid sample to affect at least partial dissolution of organic matrices or to affect ion exchange reactions.

7. The method of claim 1, wherein the pressing die system further includes a pressure injection system to facilitate introduction of a liquid component comprised of one or more reagents to the partially dehydrated biomineralized organoid sample, the pressure injection system including a pressure injection valve and a fitting that connects to the sample chamber.

8. The method of claim 7, further comprising rehydrating the biomineralized organoid sample by introduction of the liquid component via the pressure injection system.

9. The method of claim 8, wherein rehydrating the biomineralized organoid sample occurs simultaneously with mechanical compression of the biomineralized organoid sample.

10. The method of claim 8, wherein the introduced liquid component includes one or more of an aqueous liquid solution, an organic liquid solution, a gel, or a deep eutectic solvent.

11. The method of claim 8, further comprising automatically readjusting an internal pressure of the sample chamber to accommodate for introduction of the liquid component.

12. The method of claim 8, wherein the introduced liquid component includes a reagent solute to at least partially digest cellular membranes of the biomineralized organoid sample, thereby releasing and concentrating the mineral component from the biomineralized organoid sample for compression and alignment.

13. The method of claim 12, wherein the reagent solute includes an enzyme.

14. The method of claim 1, further comprising ultrasonically agitating the biomineralized organoid sample to promote fracturing cell walls of the biomineralized organoid sample so as to enhance separation of clusters of particles of the mineral component and to enhance movement of particles of the mineral component, thereby facilitating realignment of the particles in a structural arrangement.

15. The method of claim 14, wherein the structural arrangement of the particles of the mineral component exists along an axis, whereby groups of particles are aligned in a generally parallel relationship.

16. The method of claim 14, wherein the particles of the mineral component include hydroxyapatite nanocrystals.

17. The method of claim 14, wherein ultrasonic agitation of the biomineralized organoid sample includes placing at least the sample chamber containing the biomineralized organoid sample in an ultrasonic bath.

18. The method of claim 14, wherein ultrasonic agitation of the biomineralized organoid sample occurs simultaneously with a thermal treatment to increase a temperature or temperature gradient of the biomineralized organoid sample.

19. The method of claim 14, wherein ultrasonic agitation of the biomineralized organoid sample occurs simultaneously with mechanical compression of the biomineralized organoid sample.

20. The method of claim 1, wherein removal of the portion of the liquid fraction includes removal of a portion of an organic phase of the biomineralized organoid sample.

21. The method of claim 20, wherein, following removal of the portion of the organic phase, a remaining portion of the organic phase comprises approximately 1 wt % to approximately 5 wt % of the biomineralized organic sample.

22. The method of claim 20, further comprising mechanically compressing, via the force exerted by the push rod, a remaining portion of the organic phase into thin layers capable of entering into alignment with particles of the mineral component.

23. The method of claim 22, wherein the thin layers of the organic phase are intercalated with groups of particles of the mineral component in a generally parallel relationship, thereby facilitating enhanced crack resistance of a resultant mineral-based compound.

24. The method of claim 1, further comprising increasing a scale of the biomineralized organoid sample in the sample chamber to support a corresponding increase in production of a resultant mineral-based compound that exhibits enhanced density and structural alignment.

25. The method of claim 1, wherein the pressing die system utilizes a cube-shaped chamber and push-rod to facilitate formation of a mineral-based compound in the general shape of a cube.

26. The method of claim 1, wherein the pressing die system utilizes a cylinder-shaped chamber to facilitate formation of a mineral-based compound in the general shape of a cylinder.

27. A method of vacuum densification and simultaneous alignment of mineral components formed inside biomineralized organoids, the method comprising:
providing a pressing die system that includes a push rod arranged within a sleeve, a sample chamber, a vacuum pump system, and a pressure injection system connected to the sample chamber;
inserting a hydrated biomineralized organoid sample, including a mineral component, into the sample chamber;
mechanically compressing the biomineralized organoid sample, by exerting a force via the push rod, so as to partially dehydrate the biomineralized organoid sample and at least partially compact a solid fraction thereof;
rehydrating the biomineralized organoid sample by introduction of a liquid component via the pressure injection system, the liquid component including a reagent solute to at least partially digest cellular membranes of the biomineralized organoid sample, thereby releasing the mineral component from the biomineralized organoid sample;
ultrasonically agitating the biomineralized organoid sample to promote separation of clusters of particles of the mineral component and to enhance movement of particles of the mineral component, thereby enhancing alignment of the particles in a structural arrangement;
removing at least a portion of a liquid fraction from the pressing die system, via the vacuum pump system, the liquid fraction including at least a portion of an organic phase removed from the biomineralized organoid sample and at least a portion of the liquid component introduced via the pressure injection system;
heating the biomineralized organoid sample, via a controlled process using an optimized heating rate, to promote crystallization of the mineral component; and
optionally repeating one or more of the mechanical compression step, the rehydration step, the ultrasonic agitation step, the liquid fraction removal step, and the controlled heating step by one or more repetitions;
wherein the mechanical compression step, the rehydration step, the ultrasonic agitation step, the liquid fraction removal step, and the controlled heating step, alone or in any combination with one another, facilitate one or more of densification of the mineral component, alignment of particles of the mineral component in a structural arrangement, enhancement of crystallization of the mineral component, and intercalation of groups of particles of the mineral component with layers of a remaining portion of the organic phase, thereby promoting formation of a densified and structurally-aligned mineral-based compound exhibiting enhanced strength and crack resistance.

28. The method of claim 27, wherein at least two of the mechanical compression step, the rehydration step, the ultrasonic agitation step, the liquid fraction removal step, the controlled heating step, or an optional repetition of any of the foregoing steps, occur simultaneously with one another.

29. The method of claim 27, wherein the biomineralized organoid sample is an enamel organoid sample.

30. The method of claim 27, wherein the particles of the mineral component include hydroxyapatite nanocrystals.

31. The method of claim 27, wherein the pressing die system is configured so that the force generates an increasing degree of pressure upon the biomineralized organoid sample.

32. The method of claim 27, wherein the introduced liquid component includes one or more of an aqueous liquid solution, an organic liquid solution, a gel, or a deep eutectic solvent.

33. The method of claim 27, further comprising automatically readjusting an internal pressure of the sample chamber to accommodate for introduction of the liquid component.

34. The method of claim 27, wherein the reagent solute includes an enzyme.

35. The method of claim 27, wherein the structural arrangement of the particles of the mineral component exists along an axis.

36. The method of claim 35, wherein the structural arrangement includes groups of particles of the mineral component arranged in a generally parallel relationship with one another.

37. The method of claim 36, wherein mechanical compression of the biomineralized sample includes compressing the remaining portion of the organic phase into thin layers.

38. The method of claim 37, wherein one or more of the rehydration step, the ultrasonic agitation step, and the liquid fraction removal step, in combination with one another, facilitate arrangement of the thin layers into a generally parallel, intercalated relationship with the groups of particles of the mineral component.

39. The method of claim 27, wherein the remaining portion of the organic phase comprises approximately 1 wt % to approximately 5 wt % of the biomineralized organic sample.

40. The method of claim 27, wherein ultrasonically agitating the biomineralized organoid includes placing at least the sample chamber containing the biomineralized organoid in an ultrasonic bath.

41. The method of claim 27, wherein ultrasonic agitation of the biomineralized organoid sample occurs simultaneously with a thermal treatment to increase a temperature or temperature gradient of the biomineralized organoid sample.

42. The method of claim 27, wherein removal of the portion of the organic phase occurs prior to mechanical compression of the biomineralized mineral sample.

43. The method of claim 27, further comprising increasing a scale of the biomineralized organoid sample in the sample chamber to support a corresponding increase in production of a resultant mineral-based compound that exhibits enhanced density and structural alignment.

44. The method of claim 27, wherein the pressing die system utilizes a cube-shaped chamber and push-rod to facilitate formation of a mineral-based compound in the general shape of a cube.

45. The method of claim 27, wherein the pressing die system utilizes a cylinder-shaped chamber to facilitate formation of a mineral-based compound in the general shape of a cylinder.

46. The method of claim 27, further comprising heating the densified and structurally-aligned mineral-based compound to remove additional organic layers.

47. The method of claim 46, further comprising pressure injecting the densified and structurally-aligned mineral-based compound with a nutrient-rich solution, thereby filling voids left by the removed organic layers and imparting the densified and structurally-aligned mineral-based compound with an enhanced characteristic attributable to the nutrient-rich solution.

48. The method of claim 27, wherein the densified and structurally-aligned mineral-based compound includes an organic component that comprises less than 10 wt % of the compound.

49. The method of claim 27, wherein the densified and structurally-aligned mineral-based compound includes an organic component that comprises less than 3 wt % of the compound.

50. The method of claim 27, wherein the densified and structurally-aligned mineral-based compound includes an organic component that comprises less than 1 wt % of the compound.
